(19) United States
(12) Patent Application Publication
(10) Pub. No.: US 2024/0228731 A1
(43) Pub. Date: Jul. 11, 2024

(54) PROCESS FOR DEGRADING A PLASTIC PRODUCT COMPRISING AT LEAST ONE POLYESTER

(71) Applicant: CARBIOS, CLERMONT-FERRAND (FR)

(72) Inventors: ALAIN MARTY, TOULOUSE (FR); VINCIANE BORSENBERGER, TOULOUSE (FR); VINCENT TOURNIER, TOULOUSE (FR)

(21) Appl. No.: 18/560,943

(22) PCT Filed: May 20, 2022
(86) PCT No.: PCT/EP2022/063796

§ 371 Date: Nov. 15, 2023

(30) Foreign Application Priority Data
May 20, 2021 (EP) 21174995.7

(51) Int. Cl. C08J 11/10; C08J 3/12; C08J 3/20; C08J 9/00

(52) U.S. Cl. CPC C08J 11/105; C08J 3/12; C08J 3/203; C08J 9/0066; C08J 2367/02

(57) ABSTRACT

The present invention relates to a process for degrading plastic products that comprises a step of enzymatic depolymerization implemented in acidic conditions at a pH between 4 and 6, in a reaction medium containing a defined amount of soluble equivalent terephthalic acid mostly in the form of salts.

### TECHNICAL FIELD

The present invention relates to a process for degrading polyester containing material such as plastic products at an industrial or semi-industrial scale, wherein said plastic products are selected from plastic and/or textiles comprising polyester comprising at least a terephthalic acid monomer. The process of the invention particularly comprises a step of enzymatic depolymerization implemented in acidic conditions at a pH between 4 and 6, in a reaction medium containing a defined amount of soluble equivalent terephthalic acid mostly in the form of salts. Preferably, the depolymerization step is preceded by a preliminary enzymatic depolymerization step implemented at a given regulated pH comprised between 6.5 and 10. The process of the invention is particularly useful for degrading a plastic product comprising polyethylene terephthalate. The invention also relates to a process for producing monomers and/or oligomers from plastic products comprising polyester comprising at least one terephthalic acid monomer.

### BACKGROUND

Plastics are inexpensive and durable materials, which can be used to manufacture a variety of products that find uses in a wide range of applications (food packaging, textiles, etc.). Therefore, the production of plastics has increased dramatically over the last decades. Moreover, most of them are used for single-use disposable applications, such as packaging, agricultural films, disposable consumer items or for short-lived products that are discarded within a year of manufacture. Because of the durability of the polymers involved, substantial quantities of plastics are piling up in landfill sites and in natural habitats worldwide, generating increasing environmental problems. For instance, in recent years, polyethylene terephthalate (PET), an aromatic polyester produced from terephthalic acid and ethylene glycol, has been widely used in the manufacture of several products for human consumption, such as food and beverage packaging (e.g.: bottles, convenience-sized soft drinks, pouches for alimentary items) or textiles, fabrics, rugs, carpets, etc.

Different solutions, from plastic degradation to plastic recycling, have been studied to reduce environmental and economic impacts correlated to the accumulation of plastic waste. Mechanical recycling technology remains the most-used technology, but it faces several drawbacks. Indeed, it requires an extensive and costly sorting and it leads to downgrading applications, due to an overall loss of molecular weight during the process and uncontrolled presence of additives in the recycled products. The current recycling technologies are also expensive. Consequently, recycled plastic products are generally non-competitive compared to virgin plastic.

Recently, innovative processes of enzymatic recycling of plastic products have been developed and described (e.g. WO 2014/079844, WO 2015/097104, WO 2015/173265, WO 2017/198786, WO 2020/094661, and WO 2020/094646). Contrary to traditional recycling technologies, such enzymatic depolymerization processes remove the need for expensive sorting and allow for the recovery of the chemical constituents of the polymer (i.e. monomers and/or oligomers). The resulting monomers/oligomers may be recovered, purified and used to re-manufacture plastic items with equivalent quality to virgin plastic items, so that such processes lead to an infinite recycling of plastics. These processes are particularly useful for recovering terephthalic acid and ethylene glycol from plastic products comprising PET. In these processes, the production of said monomers and/or oligomers, and in particular the production of terephthalic acid, causes a decrease in the pH of the reaction medium which may be detrimental for the degrading enzyme activity. To maintain the pH and thereby an optimum enzyme activity, bases are used massively. However, to recover terephthalic acid by precipitation, a strong acid is used leading to a huge production of salts which are hardly valuable. In addition, use of base and acid as well as the lack of valorisation of the salts significantly impact the cost of these processes.

By working on this issue, the inventors have developed an optimized enzymatic process of degradation of such plastic products, which requires low addition of base and acid (and leads to low formation of salt) during the process, while maintaining a depolymerization yield satisfactory from economical and industrial point of view.

### SUMMARY OF THE INVENTION

By working on improvements of processes for degrading polyester containing material, such as plastic products, the inventors have discovered that it is possible to implement a depolymerization step under acidic conditions, in a reaction medium, while being at (or over) the saturated concentration of soluble equivalent terephthalic acid (TA) mostly in the form of salts. The use of such reaction medium exempts the operator from regulating the pH during the acidic depolymerization step.

It is thus the merit of the inventors to have determined the specific conditions enabling a good balance between base consumption and depolymerization yield acceptable at industrial scale. Particularly, the inventors have determined a saturation concentration of equivalent TA to be reached before the acidic depolymerization step to ensure an acid pH between 4 and 6 during said acidic depolymerization step. This advantageously removes the need for any pH regulation during the acidic depolymerization step and therefore base consumption.

In this regard, it is an object of the invention to provide a process for degrading a polyester containing material, such as a plastic product, comprising at least one polyester comprising at least a terephthalic acid monomer (TA) wherein the process comprises a main step of enzymatic depolymerization of said at least one polyester performed at a pH between 4 and 6, and wherein said main step of enzymatic depolymerization is implemented in a reaction medium wherein the equivalent TA concentration in the liquid phase of said reaction medium is of at least 10 g/kg, preferably of at least 20 g/kg, more preferably of at least 30 g/kg based on the total weight of the liquid phase of the reaction medium, and preferably at most of 80 g/kg, and wherein at least 90% of the equivalent TA in the liquid phase of said reaction medium is in the form of salts, preferably at least 95%, more preferably at least 99%.

It is also an object of the invention to provide a process for degrading a plastic product comprising at least one polyester comprising at least a terephthalic acid monomer (TA) in a reaction medium, wherein the process comprises a preliminary depolymerization step of said at least one polyester, preferably a preliminary enzymatic depolymerization step, performed at a given pH between 6.5 and 10, and a main step of enzymatic depolymerization performed at a pH between 4 and 6.

Preferably, the pH of the preliminary depolymerization step is regulated at said given pH by addition of base, and the pH regulation is stopped when the equivalent TA concentration in the liquid phase of the reaction medium reaches at least 5 g/kg, preferably at least 15 g/kg, more preferably at least 25 g/kg based on the total weight of the liquid phase of the reaction medium and preferably at most 110 g/kg, more preferably at most 100 g/kg.

Preferably, the process of the invention comprises:
a. A preliminary depolymerization step implemented at a given pH regulated between 7.5 and 8.5, and at a temperature between 60° and 72° C.; and
b. A main depolymerization step implemented at a pH between 5.0 and 5.5, wherein the pH is not regulated and at a temperature between 50° and 65° C.
   wherein each depolymerization step comprises contacting the plastic product with at least an enzyme able to degrade said polyester, and wherein the pH regulation of step (a) is stopped as the equivalent TA concentration in the liquid phase of the reaction medium is comprised between 5 g/kg and 110 g/kg, preferably between 30 g/kg and 100 g/kg, more preferably between 30 g/kg and 95 g/kg based on the total weight of the liquid phase of the reaction medium.

It is another purpose of the present invention to provide a reaction medium suitable to be used in a degradation process of a plastic product comprising at least one polyester comprising at least one monomer of terephthalic acid (TA), said reaction medium comprising at least 10 g/kg, preferably at least 20 g/kg, more preferably at least 30 g/kg of equivalent TA in the liquid phase of the reaction medium based on the total weight of the liquid phase of the reaction medium with at least 90%, preferably at least 95%, more preferably at least 99%, of said equivalent TA in the form of salts, and optionally at least one enzyme able to degrade a polyester.

### DETAILED DESCRIPTION OF THE INVENTION

### Definitions

In the context of the invention, a “polyester containing material” or “polyester containing product” refers to a product, such as a plastic product, comprising at least one polyester in crystalline, semi-crystalline or totally amorphous form. In a particular embodiment, the polyester containing material refers to any item made from at least one plastic material, such as plastic sheet, tube, rod, profile, shape, film, massive block, fiber, etc., which contains at least one polyester, and possibly other substances or additives, such as plasticizers, mineral or organic fillers. In another particular embodiment, the polyester containing material refers to a plastic compound, or plastic formulation, in a molten or solid state, suitable for making a plastic product. In another particular embodiment, the polyester containing material refers to textile, fabrics or fibers comprising at least one polyester. In another particular embodiment, the polyester containing material refers to plastic waste or fiber waste comprising at least one polyester. Particularly, the polyester containing material is a plastic product.

Within the context of the invention, the terms “plastic article” or “plastic product” are used to refer to any item or product comprising at least one polymer, such as plastic sheet, tube, rod, profile, shape, massive block, fiber, etc. Preferably, the plastic article is a manufactured product, such as rigid or flexible packaging (bottle, trays, cups, etc.), agricultural films, bags and sacks, disposable items or the like, carpet scrap, fabrics, textiles, etc. The plastic article may contain additional substances or additives, such as plasticizers, minerals, organic fillers or dyes. In the context of the invention, the plastic article may comprise a mix of semi-crystalline and/or amorphous polymers and/or additives.

A “polymer” refers to a chemical compound or mixture of compounds whose structure is constituted of multiple repeating units (i.e. “monomers”) linked by covalent chemical bonds. Within the context of the invention, the term “polymer” refers to such chemical compound used in the composition of a plastic product.

The term “polyester” refers to a polymer that contains the ester functional group in their main chain. Ester functional group is characterized by a carbon bound to three other atoms: a single bond to a carbon, a double bond to an oxygen, and a single bond to an oxygen. The singly bound oxygen is bound to another carbon. According to the composition of their main chain, polyesters can be aliphatic, aromatic or semi-aromatic. Polyester can be homopolymer or copolymer. As an example, polyethylene terephthalate is a semi-aromatic copolymer composed of two monomers: terephthalic acid and ethylene glycol.

The term “depolymerization”, in relation to a polymer or plastic article containing a polymer, refers to a process by which the polymer or at least one polymer of said plastic article is depolymerized and/or degraded into smaller molecules, such as monomers and/or oligomers and/or any degradation products.

According to the invention, “oligomers” refer to molecules containing from 2 to about 20 monomer units. As an example, oligomers retrieved from PET include methyl-2-hydroxyethyl terephthalate (MHET) and/or bis(2-hydroxyethyl) terephthalate (BHET) and/or 1-(2-hydroxyethyl) and/or 4-methyl terephthalate (HEMT) and/or dimethyl terephthalate (DMT).

The terms «equivalent terephthalic acid» or «equivalent TA» are used to designate any form of a molecule of terephthalic acid, i.e.
the acid form of terephthalic acid (TAH₂) corresponding to the molecule of terephthalic acid alone, i.e C₈H₆O₄,
a molecule of terephthalic acid associated with one or several cations such as sodium, potassium, ammonium, hydronium (TAH⁻, TA²⁻) to form a salt of terephthalic acid (herein after “TA salt”),
a molecule of terephthalic acid contained in an oligomer (and thereby associated with other monomers), such as MHET. Said oligomer may be in the form of salts, i.e associated with one or several cations (herein after “oligomer salt”).

The term equivalent TA does not contemplate the TA monomer(s) contained in the polymer object of the degradation process.

In an embodiment of the invention, the equivalent TA is fully in the form of salts, i.e. the equivalent TA corresponds to TA salts and/or oligomer salts.

According to the invention, the “equivalent terephthalic acid concentration” or “equivalent TA concentration” in the liquid phase of a reaction medium refers to the amount of solubilized equivalent TA measured in said liquid phase, including e.g, solubilized TAH₂; TA part of soluble TA salt (TAH⁻, TA²⁻), TA part of soluble MHET or other soluble oligomers (including oligomers in the form of salts). The equivalent TA concentration can be measured by any means known by one skilled in the art, particularly by HPLC. The equivalent TA concentration is expressed in g of equivalent TA per kg of the liquid phase of the reaction medium (g/kg), based on the total weight of the liquid phase of the reaction medium.

The term “reaction medium” refers to all the elements and compounds (including liquid, enzymes, polyester, monomers and oligomers resulting from the depolymerization of said polyester) present in a reactor during a depolymerization step, also referred as the reactor content.

According to the invention, the “liquid phase of the reaction medium” refers to the reaction medium free of any solid and/or suspended particles. Said liquid phase includes the liquid and all compounds dissolved within (including enzymes, monomers, salts, etc.). This liquid phase can be separated from the solid phase of the reaction medium and retrieved, using means known by one skilled in the art, such as filtration, decantation, centrifugation, etc. In the context of the invention, the liquid phase is notably free of residual polyester (i.e., non-degraded and insoluble polyester) and of precipitated monomers.

### Process of the Invention

By working on the optimisation of enzymatic degrading process of plastic products, the inventors have discovered that it is possible to avoid coproducts (salts) production and to improve the economic return of a plastic product degrading process by reducing the base consumption while maintaining an enzymatic activity compatible with industrial performances. More particularly, the inventors have discovered that an enzymatic depolymerization of polyester may be performed at an acid pH, without addition of any base, when the reaction medium already contains a certain amount of equivalent terephthalic acid in the form of salts. The inventors have thus developed a process wherein an acidic enzymatic depolymerization step is performed in a reaction medium comprising a defined equivalent terephthalic acid concentration mainly in the form of salts. Advantageously, said acidic depolymerization step is implemented without any regulation of pH in the reaction medium. Advantageously, said process comprises a preliminary step, prior to the acidic depolymerization step, allowing to reach said defined equivalent terephthalic acid concentration in the reaction medium.

Thus, it is an object of the invention to provide a process for degrading a polyester containing material, such as a plastic product, comprising at least one polyester comprising at least a terephthalic acid monomer (TA) wherein the process comprises a main step of enzymatic depolymerization of said at least one polyester performed at a pH between 4 and 6, and wherein said enzymatic depolymerization step is implemented in a reaction medium wherein the equivalent TA concentration in the liquid phase of said reaction medium is of at least 10 g/kg, preferably of at least 20 g/kg, more preferably of at least 30 g/kg based on the total weight of the liquid phase of the reaction medium with at least 90% of said equivalent TA is in the form of salts.

Preferably, at least 95% of the equivalent TA in the liquid phase of said reaction medium is in the form of salts, more preferably at least 96%, 97%, 98%, 99%.

### Main Depolymerization Step

According to the present invention, the main depolymerization step (also referred as “acidic depolymerization step”) is performed at a pH between 4 and 6.

The reaction medium of the main depolymerization step comprises at least a plastic product comprising at least one polyester comprising at least one monomer of TA, a liquid, at least one enzyme able to degrade said at least one polyester and a defined equivalent TA concentration in the liquid phase, mostly in the form of salts.

Advantageously, the pH of the main depolymerization step is not regulated, i.e. no base is added in the reaction medium to maintain the pH during the main depolymerization step.

Indeed, the inventors have discovered that once the reaction medium reaches a specific equivalent TA concentration, mostly in the form of salts, the pH in the reaction medium is maintained automatically (i.e., without the need of any specific action to maintain said pH) due to a physicochemical equilibrium related to the maximum concentration of TA in its acid form (TAH₂) in solution before precipitation. In such acidic conditions, wherein the liquid phase of the reaction medium is saturated in TA, any additional terephthalic acid precipitates and thus is insoluble. Consequently, during the acidic depolymerization step, any terephthalic acid produced that precipitate in the reaction medium does not impact the pH of the reaction medium.

According to the invention, the main depolymerization step is implemented at a pH between 4 and 6. Preferably, the main depolymerization step is implemented at a constant pH, or target pH, comprised between 4 and 6. In the context of the invention “a constant pH” refers to a given pH+/−0.2, preferably a given pH+/−0.1, more preferably +/−0.05.

Preferably the main depolymerization step is implemented at a pH between 4 and 5.5, more preferably at a pH between 4.5 and 5.5, even more preferably between 5 and 5.5. Particularly, the main depolymerization step is implemented at pH 5.2+/−0.2, preferably at pH 5.2+/−0.1. Alternatively, the main depolymerization step is implemented at pH 5.3+/−0.2, preferably at pH 5.3+/−0.1. Alternatively, the main depolymerization step is implemented at pH 5.4+/−0.1, alternatively at pH 5.45+/−0.05.

According to the invention, the main depolymerization step is implemented at a temperature between 40° C. and 80° C., preferably between 50° C. and 72° C., more preferably between 50° C. and 65° C., even more preferably between 50° C. and 60° C. In an embodiment, the main depolymerization step is implemented between 55° C. and 60° C. or between 50° C. and 55° C. In another embodiment, the main depolymerization step is implemented between 55° C. and 65° C. In another embodiment, the main depolymerization step is implemented between 60° C. and 72° C., preferably between 60° C. and 70° C. In an embodiment, the main depolymerization step is implemented at 60° C., +/−1° C. In another embodiment, the main depolymerization step is implemented at 56° C., +/−1° C. In an embodiment, the temperature of the main depolymerization step is maintained below the Tg of the polyester of interest. Within the context of the invention, the “polyester of interest” refers to the polyester comprising at least a terephthalic acid monomer (TA) targeted by the degradation process. Advantageously, the temperature is maintained at a given temperature +/−1° C.

In an embodiment, the main depolymerization step is implemented at a pH between 5.0 and 5.5 and at a temperature between 50° C. and 65° C.

According to the invention, the main depolymerization step is performed by contacting the plastic product with an enzyme able to degrade said polyester (such as enzymes belonging to class EC:3.1.1). In a preferred embodiment, the enzyme is a depolymerase, more preferably an esterase, even more preferably a lipase or a cutinase.

The main depolymerization step is implemented in a reaction medium wherein the equivalent TA concentration in the liquid phase of said reaction medium is of at least 10 g/kg, preferably of at least 20 g/kg, more preferably of at least 30 g/kg and at most of 80 g/kg, more preferably at most 70 g/kg based on the total weight of the liquid phase of the reaction medium, and wherein at least 90% of the equivalent TA in the liquid phase of said reaction medium is in the form of salts, preferably, at least 95%, more preferably at least 96%, 97%, 98%, 99%.

In an embodiment, the main depolymerization step is implemented in a reaction medium wherein the equivalent TA concentration in the liquid phase of said reaction medium is comprised between 20 g/kg and 80 g/kg, preferably comprised between 30 g/kg and 80 g/kg, more preferably comprised between 30 g/kg and 70 g/kg, and wherein at least 90% of the equivalent TA in the liquid phase of said reaction medium is in the form of salts, preferably, at least 95%, more preferably at least 96%, 97%, 98%, 99%.

In order to reach at least 90%, preferably at least 95%, 96%, 97%, 98%, 99%, of equivalent TA in the liquid phase of the reaction medium in the form of salts, base may be introduced in the reaction medium before implementation of the main depolymerization step in order to form, with TA or oligomer, TA salts (or oligomer salts). Any base known by one skilled in the art may be used. Particularly, the base is selected from the group consisting in sodium hydroxide (NaOH), potassium hydroxide (KOH) or ammonia (NH₄OH). Advantageously, the base is sodium hydroxide (NaOH).

In an embodiment, the equivalent TA concentration in the liquid phase of the reaction medium is comprised between 10 g/kg and 80 g/kg with at least 90% of said equivalent TA in the form of salts, and the main depolymerization step is implemented at a pH comprised between 5 and 5.5.

In an embodiment, the equivalent TA concentration in the liquid phase of the reaction medium is comprised between 10 g/kg and 60 g/kg, preferably between 20 g/kg and 50 g/kg, more preferably between 30 g/kg and 50 g/kg, with at least 90% of said equivalent TA in the form of salts and the main depolymerization step is implemented at a pH 5.25+/−0.1.

In another embodiment, the equivalent TA concentration in the liquid phase of the reaction medium is comprised between 30 g/kg and 80 g/kg, preferably between 50 g/kg and 80 g/kg, with at least 90% of said equivalent TA in the form of salts and the main depolymerization step is implemented at a pH 5.45+/−0.05.

In an embodiment, the main depolymerization step is implemented at a pH between 5.0 and 5.5, and the equivalent TA concentration in the liquid phase of the reaction medium is comprised between 5 g/kg and 110 g/kg, preferably between 30 g/kg and 100 g/kg, with at least 90% of said equivalent TA in the form of salts. In a particular embodiment, the main depolymerization step is implemented at a temperature between 50° C. and 65° C.

In a particular embodiment, additional polyester(s) and/or enzymes are added in the reaction medium once or several times during the main depolymerization step.

### Preliminary Depolymerization Step

In an embodiment, the reaction medium for the main depolymerization step is obtained by implementing a preliminary depolymerization step, prior to the main depolymerization step, performed at a given pH between 6.5 and 10, by contacting the plastic product with a depolymerizing agent in an initial reaction medium. According to the invention, the preliminary step comprises contacting the plastic product with a depolymerizing agent, selected from chemical and/or biological depolymerizing agent. Accordingly, the initial reaction medium (i.e. the reaction medium before the preliminary depolymerization step) comprises at least one plastic product comprising at least one polyester comprising at least one TA monomer, a liquid and at least one depolymerizing agent. Advantageously, said initial reaction medium is deprived of equivalent TA.

The purpose of this preliminary degradation step is to degrade at least partially a polyester of the plastic product, comprising at least a TA monomer, in order to reach the envisioned equivalent TA concentration in the reaction medium required to implement the main depolymerization step.

In an embodiment, the depolymerizing agent used for said preliminary degradation step is a biological depolymerizing agent.

Preferably, the preliminary depolymerization step is an enzymatic depolymerization step implemented by contacting the plastic product with at least one enzyme able to degrade the polyester of the plastic product. Preferably, the depolymerizing agent is a depolymerase, more preferably an esterase, even more preferably a lipase or a cutinase.

During said preliminary depolymerization step, the pH of the reaction medium is regulated at a given pH, +/−0.5, by addition of a base. Any base known by one skilled in the art may be used. Particularly, the pH may be regulated by addition in the reaction medium of a base selected from the group consisting in sodium hydroxide (NaOH), potassium hydroxide (KOH) or ammonia (NH₄OH). Advantageously, the base is sodium hydroxide (NaOH). Preferably, the pH is regulated at a given pH+/−0.1, preferably +/−0.05. That is to say that bases are added in the reaction medium in amounts required to prevent any decrease of the pH below said given pH, +/−0.1, preferably +/−0.05. The regulation of pH during said preliminary depolymerization step leads to the production of TA salts and/or oligomer salts in the reaction medium, therefore leading to at least 90% of the equivalent TA in the form of salts, preferably at least 95%, 96%, 97%, 98%, 99%.

In an embodiment, the given pH of the preliminary enzymatic depolymerization step is between 6.50 and 10.00, preferably between 7.00 and 9.50, more preferably between 7.00 and 9.00, even more preferably between 7.50 and 8.50. In a preferred embodiment, the given pH is above 7.00, preferably above 7.50, more preferably is pH 8.00+/−0.1.

In a particular embodiment, the preliminary depolymerization step is performed by use of at least one degrading enzyme and the given pH is the optimum pH of said at least one enzyme, +/−0.5. The “optimum pH of an enzyme” refers to the pH at which the enzyme exhibits the highest degradation rate at given conditions of temperature and in a given medium. Advantageously, the optimum pH of the enzyme is the optimum pH of the enzyme in the initial reaction medium.

In an embodiment, the preliminary depolymerization step is implemented at a temperature between 50° C. and 80° C., preferably between 55° C. and 75° C., between 55° C. and 72° C., between 60° C. and 72° C., more preferably at 65° C., +/−5° C., preferably +/−2° C. or +/−1° C. In an embodiment, the temperature is maintained between 55° C. and 70° C., between 55° C. and 65° C., preferably at 60° C., +/−5° C., preferably +/−2° C. or +/−1° C. In an embodiment, the temperature is maintained between 60° C. and 80° C., between 65° C. and 75° C., preferably at 72° C., +/−5° C., preferably +/−2° C. or +/−1° C. In an embodiment, the preliminary depolymerization step is implemented at 60° C. +/−5° C., preferably +/−2° C. or +/−1° C. In an embodiment, the temperature of the preliminary depolymerization step is maintained below the Tg of the polyester of interest. Advantageously, the temperature is maintained at a given temperature +/−1° C.

Accordingly, it is an object of the invention to provide a process for degrading a plastic product comprising at least one polyester, wherein said process is performed in a reaction medium and comprises:
a. a preliminary depolymerization step, as described above, implemented at a given pH regulated between 6.5 and 10, +/−0.5; and
b. a main depolymerization step, as described above, implemented at a pH between 4 and 6, +/−0.5,
   wherein both depolymerization steps comprises contacting the plastic product with at least an enzyme able to degrade said polyester.

According to this embodiment, the transition from the preliminary depolymerization step to the main depolymerization step is performed by stopping the pH regulation of the preliminary depolymerization step.

Preferably, the pH of step (a) is regulated until the equivalent TA concentration in the liquid phase of the reaction medium is of at least 5 g/kg, preferably of at least 15 g/kg, more preferably of at least 25 g/kg based on the total weight of the liquid phase of the reaction medium. Preferably, the pH regulation in step (a) is stopped when the equivalent TA concentration in the reaction medium reaches at most 110 g/kg, preferably at most 100 g/kg.

In an embodiment, the pH regulation of the preliminary depolymerization step is stopped as the equivalent TA concentration in the liquid phase of the reaction medium is comprised between 15 g/kg and 110 g/kg, preferably between 30 g/kg and 100/kg, more preferably between 30 g/kg and 95 g/kg. Particularly, the pH regulation of the preliminary depolymerization step is stopped as the equivalent TA concentration in the liquid phase of the reaction medium is comprised between 30 g/kg and 40 g/kg, between 30 g/kg and 50 g/kg, between 30 g/kg and 60 g/kg, between 30 g/kg and 70 g/kg, between 30 g/kg and 80 g/kg, between 30 g/kg and 90 g/kg, between 40 g/kg and 50 g/kg, between 40 g/kg and 60 g/kg, between 40 g/kg and 70 g/kg, between 40 g/kg and 80 g/kg, between 40 g/kg and 90 g/kg, between 40 g/kg and 95 g/kg, between 50 g/kg and 60 g/kg, between 50 g/kg and 70 g/kg, between 50 g/kg and 80 g/kg, between 50 g/kg and 90 g/kg, between 50 g/kg and 95 g/kg, between 60 g/kg and 70 g/kg, between 60 g/kg and 80 g/kg, between 60 g/kg and 90 g/kg, between 60 g/kg and 95 g/kg, between 70 g/kg and 80 g/kg, between 70 g/kg and 90 g/kg, between 70 g/kg and 95 g/kg, between 80 g/kg and 90 g/kg, between 80 g/kg and 95 g/kg, between 90 g/kg and 95 g/kg.

Alternatively to the measure or supervision of the equivalent TA concentration in the reaction medium, it is possible, during the preliminary depolymerization step, to monitor the amount of base added in the reaction medium to neutralize the TA produced during said preliminary depolymerization and thereby to regulate the pH. Therefore, according to the invention, a follow-up of base addition in the reaction medium during the preliminary depolymerization step may replace the supervision of the equivalent TA concentration in said reaction medium.

Accordingly, in an embodiment, the pH of the preliminary depolymerization step is regulated (e.g., base is added during the preliminary depolymerization step) until the amount of base added in the liquid phase of the reaction medium reaches at least 2 g/kg, preferably at least 12 g/kg, based on the total weight of the liquid phase of the reaction medium. Preferably, the pH regulation of the preliminary depolymerization step is stopped when the amount of base added in the reaction medium reaches at most 65 g/kg, preferably at most 53 g/kg based on the total weight of the liquid phase of the reaction medium. In an embodiment, the pH regulation of the preliminary depolymerization step is stopped when the amount of base added in the reaction medium is comprised between 2 g/kg and 65 g/kg, preferably between 12 g/kg and 53 g/kg. In an embodiment, the pH regulation of the preliminary depolymerization step is stopped when the amount of base added in the reaction medium is comprised between 12 g/kg and 15 g/kg, between 12 g/kg and 20 g/kg, between 12 g/kg and 30 g/kg, between 12 g/kg and 40 g/kg, between 12 g/kg and 50 g/kg, between 12 g/kg and 60 g/kg, between 15 g/kg and 20 g/kg, between 15 g/kg and 30 g/kg, between 15 g/kg and 40 g/kg, between 15 g/kg and 50 g/kg, between 15 g/kg and 53 g/kg, between 15 g/kg and 60 g/kg, between 15 g/kg and 65 g/kg, between 20 g/kg and 30 g/kg, between 20 g/kg and 40 g/kg, between 20 g/kg and 50 g/kg, between 20 g/kg and 53 g/kg, between 20 g/kg and 60 g/kg, between 20 g/kg and 65 g/kg, between 30 g/kg and 40 g/kg, between 30 g/kg and 50 g/kg, between 30 g/kg and 53 g/kg, between 30 g/kg and 60 g/kg, between 30 g/kg and 65 g/kg, between 40 g/kg and 50 g/kg, between 40 g/kg and 53 g/kg, between 40 g/kg and 60 g/kg, between 40 g/kg and 65 g/kg, between 45 g/kg and 53 g/kg, between 45 g/kg and 60 g/kg, between 45 g/kg and 65 g/kg, between 50 g/kg and 53 g/kg, between 50 g/kg and 60 g/kg, between 50 g/kg and 65 g/kg, between 53 g/kg and 60 g/kg, between 53 g/kg and 65 g/kg. In a particular embodiment, the pH regulation of the preliminary depolymerization step is stopped as the equivalent TA concentration in the liquid phase of the reaction medium is comprised between 30 g/kg and 95 g/kg, particularly between 30 g/kg and 40 g/kg, between 30 g/kg and 50 g/kg, between 30 g/kg and 60 g/kg, between 30 g/kg and 70 g/kg, between 30 g/kg and 80 g/kg, between 30 g/kg and 90 g/kg, between 40 g/kg and 50 g/kg, between 40 g/kg and 60 g/kg, between 40 g/kg and 70 g/kg, between 40 g/kg and 80 g/kg, between 40 g/kg and 90 g/kg, between 40 g/kg and 95 g/kg, between 50 g/kg and 60 g/kg, between 50 g/kg and 70 g/kg, between 50 g/kg and 80 g/kg, between 50 g/kg and 90 g/kg, between 50 g/kg and 95 g/kg, between 60 g/kg and 70 g/kg, between 60 g/kg and 80 g/kg, between 60 g/kg and 90 g/kg, between 60 g/kg and 95 g/kg, between 70 g/kg and 80 g/kg, between 70 g/kg and 90 g/kg, between 70 g/kg and 95 g/kg, between 80 g/kg and 90 g/kg, between 80 g/kg and 95 g/kg, between 90 g/kg and 95 g/kg and when the amount of base added in the reaction medium is comprised between 12 g/kg and 53 g/kg, between 12 g/kg and 45 g/kg, between 12 g/kg and 38 g/kg, particularly between 12 g/kg and 15 g/kg, between 12 g/kg and 20 g/kg, between 12 g/kg and 30 g/kg, between 12 g/kg and 40 g/kg, between 12 g/kg and 50 g/kg, between 15 g/kg and 20 g/kg, between 15 g/kg and 30 g/kg, between 15 g/kg and 38 g/kg, between 15 g/kg and 40 g/kg, between 15 g/kg and 50 g/kg, between 15 g/kg and 53 g/kg, between 20 g/kg and 30 g/kg, between 20 g/kg and 38 g/kg, between 20 g/kg and 40 g/kg, between 20 g/kg and 45 g/kg, between 20 g/kg and 50 g/kg, between 20 g/kg and 53 g/kg, between 30 g/kg and 38 g/kg, between 30 g/kg and 40 g/kg, between 30 g/kg and 45 g/kg, between 30 g/kg and 50 g/kg, between 30 g/kg and 53 g/kg, between 40 g/kg and 50 g/kg, between 40 g/kg and 53 g/kg, between 45 g/kg and 50 g/kg, between 45 g/kg and 53 g/kg, between 50 g/kg and 53 g/kg.

Accordingly, in an embodiment, the pH of the preliminary depolymerization step is regulated by addition of NaOH until the amount of NaOH added in the liquid phase of the reaction medium reaches at least 2 g/kg, more preferably at least 12 g/kg based on the total weight of the liquid phase of the reaction medium. Preferably, the pH regulation of the preliminary depolymerization step is stopped when the amount of NaOH added in the reaction medium reaches at most 45 g/kg, preferably at most 38 g/kg based on the total weight of the liquid phase of the reaction medium. In an embodiment, the pH regulation of the preliminary depolymerization step is stopped when the amount of NaOH added in the reaction medium is comprised between 2 g/kg and 45 g/kg, preferably between 12 g/kg and 38 g/kg. Particularly, the base used for pH regulation is sodium hydroxide (NaOH) and the pH regulation of the preliminary depolymerization step is stopped as the equivalent TA concentration in the liquid phase of the reaction medium is comprised between 30 g/kg and 95 g/kg, and when the amount of NaOH added in the reaction medium is comprised between 12 g/kg and 38 g/kg.

Alternatively, the pH of the preliminary depolymerization step is regulated by addition of KOH until the amount of KOH added in the liquid phase of the reaction medium reaches at least 3 g/kg, more preferably at least 17 g/kg based on the total weight of the liquid phase of the reaction medium. Preferably, the pH regulation of the preliminary depolymerization step is stopped when the amount of KOH added in the reaction medium reaches at most 65 g/kg, preferably at most 53 g/kg based on the total weight of the liquid phase of the reaction medium. In an embodiment, the pH regulation of the preliminary depolymerization step is stopped when the amount of KOH added in the reaction medium is comprised between 3 g/kg and 65 g/kg, preferably between 17 g/kg and 53 g/kg.

Advantageously, the pH regulation of the preliminary depolymerization step is stopped when at least 5% of the polyester of interest introduced in the initial reaction medium is depolymerized, preferably at least 10%, more preferably at least 20%. Particularly, the pH regulation of the preliminary depolymerization step is stopped when at most 70%, preferably at most 60% of the polyester of interest introduced in the initial reaction medium is depolymerized into monomers and/or oligomers. In another embodiment, the pH regulation of the preliminary depolymerization step is stopped when at most 50%, preferably at most 40%, more preferably at most 30% of the polyester of interest introduced in the initial reaction medium is depolymerized. Particularly, the pH regulation of the preliminary depolymerization step is stopped when between 20% and 70% of the polyester of interest introduced in the initial reaction medium is depolymerized, preferably between 40% and 70%, more preferably between 50% and 60%.

In an embodiment, the regulation of the pH in the preliminary depolymerization step is stopped when the equivalent TA concentration in the liquid phase of the reaction medium is comprised between 5 g/kg and 110 g/kg, preferably between 30 g/kg and 100 g/kg and the following main depolymerization step is implemented at a pH between 5.0 and 5.5.

In an embodiment, the regulation of the pH in the preliminary depolymerization step is stopped when the equivalent TA concentration in the liquid phase of the reaction medium is comprised between 5 g/kg and 60 g/kg, preferably between 20 g/kg and 50 g/kg, more preferably between 30 g/kg and 50 g/kg, and the main depolymerization step is implemented at pH 5.25+/−0.10.

In an embodiment, the regulation of the pH in the preliminary depolymerization step is stopped when the equivalent TA concentration in the liquid phase of the reaction medium is comprised between 30 g/kg and 110 g/kg, preferably between 50 g/kg and 110 g/kg, more preferably between 50 g/kg and 95 g/kg and the main depolymerization step is implemented at pH 5.45+/−0.05.

In an embodiment, the pH regulation of the preliminary depolymerization step is stopped when the amount of NaOH added in the liquid phase of the reaction medium is comprised between 2 g/kg and 45 g/kg, and the following main depolymerization step is implemented at a pH between 5.0 and 5.5.

In an embodiment, the pH regulation of the preliminary depolymerization step is stopped when the amount of NaOH added in the liquid phase of the reaction medium is comprised between 2 g/kg and 25 g/kg, preferably between 8 g/kg and 20 g/kg, more preferably between 12 g/kg and 20 g/kg, and the main depolymerization step is implemented at pH 5.25+/−0.10.

In an embodiment, the pH regulation of the preliminary depolymerization step is stopped when the amount of NaOH added in the liquid phase of the reaction medium is comprised between 12 g/kg and 45 g/kg, preferably between 20 g/kg and 45 g/kg, more preferably between 20 g/kg and 38 g/kg and the main depolymerization step is implemented at pH 5.45+/−0.05.

In an embodiment, the process of invention comprises:
a. A preliminary depolymerization step implemented at a given pH regulated between 7.5 and 8.5, and at a temperature between 60° and 72° C.; and
b. A main depolymerization step implemented at a pH between 5.0 and 5.5, wherein the pH is not regulated and at a temperature between 50° and 65° C.,
   wherein each depolymerization step comprises contacting the plastic product with at least an enzyme able to degrade said polyester, and wherein the pH regulation of step (a) is stopped as the equivalent TA concentration in the liquid phase of the reaction medium is comprised between 5 g/kg and 110 g/kg, preferably between 30 g/kg and 100/kg, more preferably between 30 g/kg and 95 g/kg. Alternatively, the main depolymerization step implemented at a pH between 5.0 and 5.5, wherein the pH is not regulated and at a temperature between 65° and 72° C. Preferably, the pH regulation of the preliminary depolymerization step (a) is performed by addition of NaOH and said pH regulation is stopped when the amount of NaOH added in the liquid phase of the reaction medium is comprised between 2 g/kg and 45 g/kg, preferably between 5 g/kg and 40 g/kg, more preferably between 12 g/kg and 38 g/kg. In an embodiment, the pH regulation of step (a) is stopped as the equivalent TA concentration in the liquid phase of the reaction medium is comprised between 30 g/kg and 50 g/kg, and the step (b) is implemented at pH 5.25+/−0.1. Alternatively, the regulation of the pH in the step (a) is stopped when the equivalent TA concentration in the liquid phase of the reaction medium is comprised between 50 g/kg and 110 g/kg, preferably between 50 g/kg and 95 g/kg and the step (b) is implemented at pH 5.45+/−0.05.

In a particular embodiment, if the pH decreases below a target pH during the main depolymerization step, an addition of base can be done occasionally to increase the pH up to the target pH. Said target pH is advantageously defined before implementation of the main depolymerization step. Particularly, the target pH is comprised between 4 and 6, +/−0.5, preferably +/−0.2, +/−0.1.

In a particular embodiment, the process of the invention may comprise a step between the preliminary depolymerization step and the main depolymerization step, wherein a base or an acid is added in the reaction medium in order to reach the target pH of the main depolymerization step.

Alternatively, or in addition, the depolymerizing agent of the preliminary depolymerization step may be a chemical depolymerizing agent. In such case, no pH regulation is needed during the preliminary depolymerization step, and the preliminary depolymerization step is implemented until the equivalent TA concentration in the liquid phase of the reaction medium reaches at least 5 g/kg, preferably at least 15 g/kg, more preferably at least 25 g/kg based on the total weight of the liquid of the reaction medium.

According to the invention, the preliminary depolymerization step and the main depolymerization step are advantageously performed at the same temperature. In an embodiment, both steps are performed at 60° C.+/−5° C., preferably +/−2° C. or +/−1° C. In another embodiment, both steps are performed at 56° C.+/−5° C., preferably +/−2° C. or +/−1° C.

### TA Salts Addition

In another embodiment, the main depolymerization step is implemented directly by use of a reaction medium comprising the defined equivalent TA concentration (mainly in the form of salts), i.e., without performing a preliminary depolymerization step. Any means known by one skilled in the art may be used to prepare the reaction medium of the main depolymerization step comprising the defined equivalent TA concentration, said equivalent TA being mostly in the form of salts.

In an embodiment, the defined equivalent TA concentration, mostly in the form of salts, in the reaction medium may be reached by addition of TA in the form of salts (TA salts and/or oligomer salts) e.g., by addition of disodium terephthalate C₈H₄Na₂O₄, dipotassium terephthalate C₈H₄K₂O₄ diammonium terephthalate C₈H₁₂N₂O₄, monosodium terephthalate C₈H₅NaO₄, monopotassium terephthalate C₈H₅KO₄ and/or monoammonium terephthalate C₈H₁₀NO₄ in the reaction medium prior to the main depolymerization step.

Alternatively or in addition, the defined equivalent TA concentration, mostly in the form of salts, in the reaction medium may be reached by addition, in the reaction medium, of both TA in its acid form and base, to produce TA salts.

Preferably, TA salts (and/or oligomer salts and/or both TA in its acid form and base separately) are added in order to reach an equivalent TA concentration in the liquid phase of the reaction medium of at least 10 g/kg, preferably at least 20 g/kg, more preferably at least 30 g/kg based on the total weight of the liquid phase of the reaction medium prior to the main depolymerization step, and preferably of at most 80 g/kg, more preferably at most 70 g/kg, with at least 90% of the equivalent TA in the liquid phase in the form of salts.

In an embodiment, TA salts (and/or oligomer salts and/or both TA in its acid form and base separately) are added in order to reach an equivalent TA concentration in the liquid phase of the reaction medium comprised between 20 g/kg and 80 g/kg, preferably comprised between 30 g/kg and 80 g/kg, more preferably comprised between 30 g/kg and 70 g/kg with at least 90% of the equivalent TA in the liquid phase in the form of salts.

In an embodiment, TA salts (and/or oligomer salts and/or both TA in its acid form and base separately) are added in order to reach an equivalent TA concentration in the liquid phase of the reaction medium comprised between 10 g/kg and 80 g/kg, preferably between 30 g/kg and 80 g/kg with at least 90% of the equivalent TA in the liquid phase in the form of salts, and the main depolymerization step is implemented at a pH comprised between 5 and 5.5.

In an embodiment, TA salts (and/or oligomer salts and/or both TA in its acid form and base separately) are added in order to reach an equivalent TA concentration in the liquid phase of the reaction medium comprised between 10 g/kg and 60 g/kg, preferably between 20 g/kg and 50 g/kg, more preferably between 30 g/kg and 50 g/kg, with at least 90% of the equivalent TA in the liquid phase in the form of salts, and the main depolymerization step is implemented at pH 5.25+/−0.1.

In another embodiment, TA salts (and/or oligomer salts and/or both TA in its acid form and base separately) are added in order to reach an equivalent TA concentration in the liquid phase of the reaction medium comprised between 30 g/kg and 80 g/kg, preferably between 50 g/kg and 80 g/kg, with at least 90% of the equivalent TA in the liquid phase in the form of salts, and the main depolymerization step is implemented at pH 5.45+/−0.05.

In an embodiment, the TA salts and/or oligomer salts added in the reaction medium are retrieved from a previous chemical and/or enzymatic depolymerization step as defined above (or in WO 2020/094661), preferably regulated by addition of base at a pH between 6.5 and 10. The TA salts may be retrieved by using any purification methods, such as the ones described in WO 2020/094661, to be added in the reaction medium of the main depolymerization step.

In an embodiment, the reaction medium of the main depolymerization step is prepared by both an addition of extraneous TA salts (and/or oligomer salts and/or both TA in its acid form and base separately) in the reaction medium and the implementation of a preliminary depolymerization step as described above, leading to production of TA, in order to achieve the target equivalent TA concentration in the reaction medium with at least 90% of the equivalent TA in the liquid phase in the form of salts.

### Enzymes and Microorganisms

According to the invention, at least the main depolymerization step, and optionally the preliminary depolymerization step, is/are implemented by contacting the plastic product comprising at least one polyester comprising at least a TA monomer with at least an enzyme able to degrade said polyester. In an embodiment, the enzymatic depolymerization step(s) is/are implemented by contacting the plastic product comprising at least one polyester comprising at least a TA monomer with at least a microorganism that expresses and excretes said enzyme able to degrade said polyester.

In an embodiment, said at least one enzyme exhibits a polyester-degrading activity at a pH between 4 and 10, particularly between 4 and 9. In another embodiment, said at least one enzyme has an optimum pH between 6.5 and 10, particularly between 6.5 and 9, and still exhibits a polyester-degrading activity at a pH between 4 and 6, preferably at a pH between 5 and 5.5 and/or at the pH of the main depolymerization step.

In the context of the invention, a “polyester-degrading activity” can be assessed by any means known by the skilled person. Particularly, a “polyester-degrading activity” can be assessed by measurement of the specific polyester's depolymerization activity rate, the measurement of the rate to degrade a solid polyester compound dispersed in an agar plate, the measurement of the polyester's depolymerization activity rate in reactor, the measurement of the quantity of depolymerization products (EG, TA, MHET, . . . ) released, the mass measurement of the polyester.

In an embodiment, the enzyme exhibiting a polyester-degrading activity is selected from depolymerases, preferably selected from esterases. In a preferred embodiment, the enzyme is selected from lipases or cutinases.

In a particular embodiment, the enzyme is an esterase. Particularly, the esterase is a cutinase, preferably a cutinase coming from a microorganism selected from Thermobifida cellulosityca, Thermobifida halotolerans, Thermobifida fusca, Thermobifida alba, Bacillus subtilis, Fusarium solani pisi, Humicola insolens, Sirococcus conigenus, Pseudomonas mendocina, Thielavia terrestris, Saccharomonospora viridis, Thermomonospora curvata or any functional variant thereof. In another embodiment, the cutinase is selected from a metagenomic library such as LC-Cutinase described in Sulaiman et al., 2012 or the esterase described in EP3517608, or any functional variant thereof including depolymerases listed in WO 2021/005198, WO 2018/011284, WO 2018/011281, WO 2020/021116, WO 2020/021117 or WO 2020/021118. In another particular embodiment, the esterase is a lipase preferably coming from Ideonella sakaiensis or any functional variant thereof, including the lipase described in WO 2021/005199. In another particular embodiment, the depolymerase is a cutinase coming from Humicola insolens, such as the one referenced A0A075B5G4 in Uniprot or any functional variant thereof. In another embodiment, the depolymerase is selected from commercial enzymes such as Novozym 51032 or any functional variant thereof.

In another particular embodiment, the enzyme is selected from enzymes having at least 75%, 80%, 85%, 90%, 95%, 96%, 97%, 98%, or 99% identity to the full length amino acid sequence set forth in SEQ ID No 1 and/or to the full length amino acid sequence set forth in SEQ ID No 3, and exhibiting a polyester-degrading activity, particularly a PET-degrading activity.

In an embodiment, the enzyme is selected from enzymes having a PET-degrading activity (PETase) and/or enzymes having a MHET-degrading activity (MHETase).

In the context of the invention, a “MHET-degrading activity” can be assessed by any means known by the skilled person. As an example, the “MHET-degrading activity” can be assessed by measurement of the MHET degradation activity rate by the measurement of the quantity of depolymerization products (ethylene glycol EG and TA) released.

In an embodiment, the MHETase may be selected from depolymerases, preferably selected from esterases. In an embodiment, the MHETase is selected from lipases or cutinases. In another embodiment, the MHETase is selected from enzymes belonging to the class EC:3.1.1.102.

In a particular embodiment, the MHETase is selected from an MHETase isolated or derived from Ideonella sakaiensis, as disclosed in Yoshida et al., 2016, or any functional variant thereof. In another particular embodiment, the MHETase is selected from enzymes having at least 75%, 80%, 85%, 90%, 95%, 96%, 97%, 98%, or 99% identity to the full length amino acid sequence set forth in SEQ ID No 2.

In a particular embodiment, the PETase and the MHETase are included in a multienzyme system, particularly a two-enzyme system such as the Ideonella sakaiensis PETase/MHETase system disclosed in Knott et al. 2020.

In an embodiment, the enzyme is selected from enzymes having an optimum pH between 4 and 6 and/or exhibiting a polyester-degrading activity at a pH between 4 and 6.

In an embodiment the main depolymerization step and the preliminary enzymatic depolymerization step are implemented by contacting the plastic product comprising at least one polyester with at least two enzymes, preferably with at least two enzymes exhibiting said polyester degrading activity, wherein:
at least a first enzyme exhibits said polyester degrading activity at a pH between 6.5 and 10, preferably at the pH of the preliminary enzymatic depolymerization step and
at least a second enzyme, different from the first enzyme, exhibits said polyester degrading activity at a pH between 4 and 6, preferably at the pH of the main depolymerization step.

In an embodiment, both steps are implemented by contacting the plastic product comprising at least one polyester with at least two enzymes, preferably with at least two enzymes exhibiting said polyester degrading activity, wherein:
at least a first enzyme exhibits said polyester degrading activity at a pH between 6.5 and 10, preferably at the pH of the preliminary enzymatic depolymerization step and
at least a second enzyme, different from the first enzyme, exhibits said activity at a pH between 4 and 10.

In a particular embodiment, the plastic product comprises PET and both steps are implemented by contacting the plastic product comprising at least PET with at least two enzymes, preferably at least one PETase and at least one MHETase. In an embodiment, the preliminary depolymerization step is implemented by contacting the plastic product comprising at least one polyester with at least one PETase and the main depolymerization step is implemented with at least one MHETase. In an embodiment, the preliminary depolymerization step is implemented by contacting the plastic product comprising at least one polyester with at least one PETase and at least one MHETase is added during the main depolymerization step in addition to the PETase. In a particular embodiment, the preliminary depolymerization step is implemented by contacting the plastic product comprising at least one polyester with at least a PETase, and a MHETase is added during the main depolymerization step in addition to the PETase.

MHETase may be added simultaneously to PETase. Alternatively or in addition, MHETase may be added after PETase, for instance once polyester has been at least partially degraded by PETase.

In an embodiment, the plastic product is contacted simultaneously with the PETase and the MHETase. In another embodiment, the plastic product is contacted first with the PETase, and the MHETase is introduced in the reaction medium after the PETase.

The simultaneous use of a PETase and a MHETase during the preliminary depolymerization step and/or the main depolymerization step may in particular embodiments lead to a synergistic effect, thus leading to a depolymerization rate higher than the sum of the depolymerization rates obtained with the PETase alone and the MHETase alone.

In an embodiment, the enzymes used in the preliminary depolymerization step and/or in the main depolymerization step are selected from enzymes having at least 75%, 80%, 85%, 90%, 95%, 96%, 97%, 98%, or 99% identity to the full length amino acid sequence set forth in SEQ ID no 1 and/or SEQ ID no 3, and the MHETase of SEQ ID no 2.

The enzymes may be in soluble form, or solid phase such as powder form. In particular, they may be bound to cell membranes or lipid vesicles, or to synthetic supports such as glass, plastic, polymers, filter, membranes, e.g., in the form of beads, columns, plates and the like. The enzymes may be in an isolated or purified form. Preferentially, the enzymes of the invention are expressed, derived, secreted, isolated, or purified from microorganisms. The enzymes may be purified by techniques known per se in the art and stored under conventional techniques. The enzymes may be further modified to improve e.g., their stability, activity and/or adsorption on the polymer. For instance, the enzymes are formulated with stabilizing and/or solubilizing components, such as water, glycerol, sorbitol, dextrin, including maltodextrine and/or cyclodextrine, starch, propanediol, salt, etc.

In another embodiment, one or both steps of depolymerization is/are implemented with at least one microorganism that expresses and excretes the depolymerase. In the context of the invention the enzyme may be excreted in the culture medium or towards the cell membrane of the microorganism wherein said enzyme may be anchored. Said microorganism may naturally synthesize the depolymerase, or it may be a recombinant microorganism, wherein a recombinant nucleotide sequence encoding the depolymerase has been inserted, using for example a vector. For example, a nucleotide molecule, encoding the depolymerase of interest is inserted into a vector, e.g. plasmid, recombinant virus, phage, episome, artificial chromosome, and the like. Transformation of the host cell as well as culture conditions suitable for the host are well known to those skilled in the art.

The recombinant microorganisms may be used directly. Alternatively, or in addition, recombinant enzymes may be purified from the culture medium. Any commonly used separation/purification means, such as salting-out, heat shock, gel filtration, hydrophobic interaction chromatography, affinity chromatography or ion exchange chromatography may be used for this purpose. In particular embodiments, microorganisms known to synthesize and excrete depolymerases of interest may be used.

According to the invention, several enzymes and/or several microorganisms may be used together or sequentially during the different depolymerization steps.

According to the invention, the enzyme quantity in the reaction medium is comprised between 0.1 mg/g and 15 mg/g of the targeted polyester, preferably between 0.1 mg/g and 10 mg/g, more preferably between 0.1 mg/g and 5 mg/g, even more preferably between 0.5 mg/g and 4 mg/g. Preferably, the enzyme quantity in the reaction medium is at most 4 mg/g, preferably at most 3 mg/g, more preferably at most 2 mg/g of the targeted polyester. When at least one PETase and at least one MHETase are used, the PETase amount in the reaction medium is comprised between 0.1 mg/g and 10 mg/g, preferably between 0.1 mg/g and 5 mg/g, more preferably between 0.5 mg/g and 4 mg/g, of the targeted polyester and the MHETase amount in the reaction medium is comprised between 0.1 mg/g and 5 mg/g, preferably between 0.1 mg/g and 2 mg/g of the targeted polyester.

According to an embodiment of the invention, the process of invention comprises:
a. A preliminary depolymerization step implemented at a given pH regulated between 7.5 and 8.5, and at a temperature between 60° and 72° C. by contacting the plastic product with at least one PETase; and
b. A main depolymerization step implemented at a pH between 5.0 and 5.5, wherein the pH is not regulated and at a temperature between 50° and 65° C. by contacting the plastic product with at least one PETase and optionally at least one MHETase,
   wherein the pH regulation of step (a) is stopped as the equivalent TA concentration in the liquid phase of the reaction medium is comprised between 30 g/kg and 95 g/kg. Optionally, additional amounts of enzymes (PETase and/or MHETase) may be added once or several times to the reaction medium during the main depolymerization step.

According to an embodiment of the invention, the process of invention comprises:
a. A preliminary depolymerization step implemented at a given pH regulated between 7.5 and 8.5, and at a temperature between 60° and 72° C. by contacting the plastic product simultaneously with at least one PETase and at least one MHETase; and
b. A main depolymerization step implemented at a pH between 5.0 and 5.5, wherein the pH is not regulated and at a temperature between 50° and 65° C.,
   wherein the pH regulation of step (a) is stopped as the equivalent TA concentration in the liquid phase of the reaction medium is comprised between 30 g/kg and 95 g/kg. Optionally, additional amounts of enzymes (PETase and/or MHETase) may be added once or several times to the reaction medium during the main depolymerization step.

Advantageously, the PETase is selected from enzymes having at least 75%, 80%, 85%, 90%, 95%, 96%, 97%, 98%, or 99% identity to the full length amino acid sequence set forth in SEQ ID No 1 and/or to the full length amino acid sequence set forth in SEQ ID No 3, and exhibiting a polyester-degrading activity and the MHETase is selected from enzymes having at least 75%, 80%, 85%, 90%, 95%, 96%, 97%, 98%, or 99% identity to the full length amino acid sequence set forth in SEQ ID No 2.

### Chemical Depolymerization Agent

In an embodiment, the preliminary depolymerization step comprises or consists in a chemical depolymerization step, which is implemented by contacting the plastic product comprising at least one polyester with at least one chemical agent.

In an embodiment, the chemical agent is a catalyst. The chemical agent can be selected from any catalyst known by a person of the art for having the capacity to degrade and/or depolymerize the target polyester. Advantageously, the catalyst is selected from metallic catalysts or stables and not toxic hydrosilanes (PMHS, TMDS) such as commercially available B(C₆F₅)₃ and [Ph₃C⁺B(C₆F₅)⁴⁻] catalysts. Particularly, the catalyst is selected from alkoxide, carbonate, acetate, hydroxide, alkaline metal oxide, alkaline earth metal, calcium oxide, calcium hydroxide, calcium carbonate, sodium carbonate, iron oxide, zinc acetate, zeolite. In some embodiments, the catalyst comprises at least one of germanium compounds, titanium compounds, antimony compounds, zinc compounds, cadmium compounds, manganese compounds, magnesium compounds, cobalt compounds, silicon compounds, tin compounds, lead compounds, and aluminum compounds. Particularly, the catalyst comprises at least one of germanium dioxide, cobalt acetate, titanium tetrachloride, titanium phosphate, titanium tetrabutoxide, titanium tetraisopropoxide, titanium tetra-n-propoxide, titanium tetraethoxide, titanium tetramethoxide, a tetrakis(acetylacetonato)titanium complex, a tetrakis(2,4-hexanedionato)titanium complex, a tetrakis(3,5-heptanedionato)titanium complex, a dimethoxybis(acetylacetonato)titanium complex, a diethoxybis(acetylacetonato)titanium complex, a diisopropoxybis(acetylacetonato)titanium complex, a di-n-propoxybis(acetylacetonato)titanium complex, a dibutoxybis(acetylacetonato)titanium complex, titanium dihydroxybisglycolate, titanium dihydroxybisglycolate, titanium dihydroxybislactate, titanium dihydroxybis(2-hydroxypropionate), titanium lactate, titanium octanediolate, titanium dimethoxybistriethanol aminate, titanium diethoxybistriethanol aminate, titanium dibutoxybistriethanol aminate, hexamethyl dititanate, hexaethyl dititanate, hexapropyl dititanate, hexabutyl dititanate, hexaphenyl dititanate, octamethyl trititanate, octaethyl trititanate, octapropyl trititanate, octabutyl trititanate, octaphenyl trititanate, a hexaalkoxy dititanate, zinc acetate, manganese acetate, methyl silicate, zinc chloride, lead acetate, sodium carbonate, sodium bicarbonate, acetic acid, sodium sulfate, potassium sulfate, zeolites, lithium chloride, magnesium chloride, ferric chloride, zinc oxide, magnesium oxide, calcium oxide, barium oxide, antimony trioxide, and antimony triacetate.

Alternatively or in addition, the catalyst is selected from nanoparticules.

Alternatively, the chemical agent is an acid or a base catalyst that is able to break polymer bonds, particularly esters bonds. Particularly, the chemical agent involved in breaking of esters bonds is a mixture of hydroxide and an alcohol that can dissolve the hydroxide. The hydroxide is selected from alkali metal hydroxide, alkaline-earth metal hydroxide, and ammonium hydroxide, preferably selected from sodium hydroxide, potassium hydroxide, calcium hydroxide, lithium hydroxide, magnesium hydroxide, ammonium hydroxide, tetra-alkyl ammonium hydroxide and the alcohol is selected from linear, branched, cyclic alcohol or a combination thereof, preferably linear C1-C4 alcohol selected from methanol, ethanol, propanol, butanol.

In a particular embodiment, the chemical agent is a mixture of a non-polar solvent able to swell the polyester (i.e., swelling agent) and an agent that can break or hydrolyze ester bonds, wherein the swelling agent is preferably a chlorinated solvent selected from dichloromethane, dichloroethane, tetrachloroethane, chloroform, tetrachloromethane and trichloroethane. In another particular embodiment, the chemical agent is an acid selected from ethylene glycol, hydrochloric acid, sulfuric acid or a Lewis acid.

### Polyesters

In an embodiment, the process of the invention is implemented with plastic products from plastic waste collection and/or post-industrial waste. More particularly, the process of the invention may be used for degrading domestic plastic wastes, including plastic bottles, plastic trays, plastic bags, plastic packaging, soft plastics and/or hard plastics, even polluted with food residues, surfactants, etc. Alternatively, or in addition, the process of the invention may be used for degrading used plastic fibers, such as fibers providing from fabrics, textiles and/or and industrial wastes. More particularly, the process of the invention may be used with PET plastic and/or PET fiber waste, such as PET fibers coming from fabrics, textile, and/or tires.

According to the invention, the plastic product comprises at least one polyester selected from polyethylene terephthalate (PET), polytrimethylene terephthalate (PTT), polybutylene terephthalate (PBT), polyethylene isosorbide terephthalate (PEIT), polybutylene adipate terephthalate (PBAT), polycyclohexylenedimethylene terephthalate (PCT), glycosylated polyethylene terephthalate (PETG), poly (butylene succinate-co-terephtalate) (PBST), poly(butylene succinate/terephthalate/isophthalate)-co-(lactate) (PBSTIL) and blends/mixtures of these polymers, preferably selected from polyethylene terephthalate (PET).

In an embodiment, the plastic product comprises at least one amorphous polyester targeted by the degrading process.

In an embodiment, the plastic product comprises at least one crystalline polyester and/or at least one semi-crystalline polyester targeted by the degrading process. In the context of the invention, “semi-crystalline polyester” refers to partially crystalline polyester wherein crystalline regions and amorphous regions coexist. The degree of crystallinity of a semi-crystalline polyester may be estimated by different analytical methods and typically ranges from 10 to 90%. For instance, Differential Scanning calorimetry (DSC) or X-Ray diffraction may be used for determining the degree of crystallinity of polymers.

In an embodiment, the plastic product comprises crystalline polyester and/or semi-crystalline polyester, and amorphous polyester, targeted by the degrading process.

In an embodiment, the plastic article may be pretreated prior to the main depolymerization step (or prior to the preliminary depolymerization step if any) in order to physically change its structure, so as to increase the surface of contact between the polyester and the enzymes and/or to decrease the microbial charge coming from wastes. Examples of pretreatments are described in the patent application WO 2015/173265.

According to the invention, it is possible to submit the polyester of the plastic product to an amorphization step prior to the main depolymerization step (or prior to the preliminary depolymerization step if any) by any means known by one skilled in the art. An example of amorphization process is described in the patent application WO 2017/198786. In a particular embodiment, the polyester is submitted to an amorphization process followed by a granulation and/or micronization process prior any depolymerization step.

Alternatively, it is possible to submit the plastic article to a foaming step prior to the main depolymerization step (or prior to the preliminary depolymerization step if any) by any means known by one skilled in the art. An example of foaming pretreatment process is described in the patent application PCT/EP2020/087209.

In a preferred embodiment, the plastic product is pretreated prior to the main depolymerization step (or prior to the preliminary depolymerization step if any) and the polyester of interest of the plastic product exhibits a degree of crystallinity below 30% before being submitted to the main depolymerization step (or to the preliminary depolymerization step), preferably a degree of crystallinity below 25%, more preferably below 20%.

### Reactor

According to the invention, the process may be implemented in any reactor having a volume greater than 500 mL, greater than 1 L, preferably greater than 2 L, 5 L or 10 L. In a particular embodiment, the process is implemented at a semi-industrial or an industrial scale. Accordingly, the process may be implemented in a reactor having a volume greater than 100 L, 150 L, 1 000 L, 10 000 L, 100 000 L, 400 000 L.

In the context of the invention, the total volume of the reactor is advantageously at least 10% greater than the volume of the reaction medium, or reactor content.

According to the invention, the reactor content is maintained under agitation during the process. The speed of the agitation is regulated by one skilled in the art so as to be sufficient to allow the suspension of the plastic product in the reactor, the homogeneity of the temperature and the precision of the pH regulation if any.

In an embodiment, the concentration of polyester introduced before the main depolymerization step (or before to the preliminary depolymerization step if any) is above 150 g/kg in relation to the total weight of the reaction medium (or the initial reaction medium), preferably above 200 g/kg, more preferably above 300 g/kg, even more preferably above 400 g/kg.

In a particular embodiment, the concentration of polyester introduced before the main depolymerization step (or before the preliminary depolymerization step if any) is comprised between 200 g/kg and 400 g/kg, preferably between 300 g/kg and 400 g/kg. Alternatively, the concentration of polyester introduced before the main depolymerization step (or before to the preliminary depolymerization step if any) is comprised between 400 g/kg and 600 g/kg.

In a preferred embodiment, the reaction medium comprises as a liquid an aqueous solvent such as buffer and/or water, preferably water. In a preferred embodiment, the liquid in the reaction medium is free of non-aqueous solvent, especially inorganic solvent. In a particular embodiment, the liquid in the reaction medium consists in water only.

In an embodiment, during the main depolymerization step, additional amounts of polyester and/or enzymes (such as PETase and/or MHETase) may be added in the reaction medium, continuously or sequentially. Particularly, additional amounts of polyester and/or enzymes may be added, once or several times, during the main depolymerisation step.

Particularly, polyester may be added in order to reach a final concentration of polyester introduced in the reaction medium comprised between 300 g/kg and 600 g/kg of polyester, preferably between 400 g/kg and 600 g/kg, more preferably between 500 g/kg and 600 g/kg. The final concentration of polyester corresponds to the total quantity of polyester introduced during the whole degrading process in the reaction medium based on the total weight of the reaction medium before the main depolymerization step or based on the total weight of the initial reaction medium (i.e. before the preliminary depolymerization step, if any).

In an embodiment, the concentration of polyester introduced before the main depolymerization step is below 300 g/kg in relation to the total weight of the reaction medium, preferably between 200 g/kg and 300 g/kg, and further polyesters are added during the main depolymerization step in order to reach a final concentration of polyester introduced in the reaction medium above 400 g/kg, more preferably above 500 g/kg, even more preferably between 500 g/kg and 600 g/kg. In such embodiment, the main depolymerization step is preferably performed in a reaction medium at a pH between 5 and 5.5 and with an equivalent TA concentration in the liquid phase of said reaction medium comprised between 30 g/kg and 70 g/kg based on the total weight of the liquid phase of the reaction medium with at least 90% of the equivalent TA in the liquid phase of said reaction medium in the form of salts. Optionally, further enzymes are also added during the main depolymerization step.

In a particular embodiment, the process of the invention performed in a reaction medium comprises:
a. a preliminary depolymerization step implemented at a given pH regulated between 6.5 and 10, preferably between 7.5 and 8.5; and
b. a main depolymerization step implemented at a pH between 5 and 5.5,
wherein both depolymerization steps comprise contacting the plastic product with at least an enzyme able to degrade said polyester,
wherein the concentration of polyester introduced before the preliminary depolymerization step is below 300 g/kg in relation to the total weight of the initial reaction medium, preferably between 200 g/kg and 300 g/kg, and further polyester is added during the main depolymerization step in order to reach a final concentration of polyester introduced in the reaction medium, based on the total weight of the initial reaction medium, above 400 g/kg, more preferably above 500 g/kg, even more preferably between 500 g/kg and 600 g/kg,
wherein the pH of step (a) is regulated until the equivalent TA concentration in the liquid phase of the reaction medium is of at least 25 g/kg, preferably between 50 g/kg and 95 g/kg, based on the total weight of the liquid phase of the reaction medium.

Optionally, further enzymes are also added during the main depolymerization step.

It is also an object of the invention to provide a reaction medium suitable for implementing the main depolymerization step of the degradation process of the present invention, said reaction medium comprising at least 10 g/kg, preferably at least 20 g/kg, more preferably at least 30 g/kg of equivalent TA in the liquid phase based on the total weight of the liquid phase of the reaction medium, with at least 90%, preferably at least 95%, 96%, 97%, 98%, 99%, of said equivalent TA in the form of salts. Preferably, the reaction medium comprises at most 80 g/kg of equivalent TA in the liquid phase based on the total weight of the liquid phase of the reaction medium.

### Purification

In a particular embodiment, the process for degrading polymer containing material, such as a plastic product, further comprises a step of recovering and optionally purifying the monomers and/or oligomers and/or degradation products, preferably terephthalic acid, resulting from the step(s) of depolymerization. Monomers and/or oligomers and/or degradation products resulting from the depolymerization may be recovered, sequentially or continuously.

A single type of monomers and/or oligomers or several different types of monomers and/or oligomers may be recovered. The recovered monomers and/or oligomers and/or degradation products may be purified, using all suitable purifying method and optionally conditioned in a re-polymerizable form. An example of purification is described in the patent application WO 1999/023055. In a particular embodiment, the recovery of TA under solid form comprises separating the solid phase from the liquid phase of the reaction medium by filtration.

The solid phase recovered may be dissolved and/or dispersed in a solvent selected from water, DMF, NMP, DMSO, DMAC or any solvent known to solubilized TA, and filtered to remove impurities. Solubilized TA can then be recrystallized by any means known by one skilled in the art.

The TA salts contained in the liquid phase of the reaction medium can be recovered to be reused in another process of degradation according to the invention in order to reach the defined equivalent TA concentration in the liquid phase of the reaction medium of said other degradation process.

In an embodiment, after the main depolymerization step, a MHETase is added in the reaction medium before the purification process, in order to hydrolyze the MHET produced during the depolymerization step(s) to produce TA.

In a preferred embodiment, the repolymerizable monomers and/or oligomers may then be reused to synthesize polymers. One skilled in the art may easily adapt the process parameters to the monomers/oligomers and the polymers to synthesize.

Accordingly, it is also an object of the invention to provide a process for recycling polyester containing material, such as a plastic article, comprising at least one polyester comprising at least one TA monomer, preferably PET, and/or to provide a method of producing monomers and/or oligomers and/or degradation products, preferably TA, from a plastic article comprising at least one polyester having at least one TA monomer, comprising submitting the plastic article to a main enzymatic depolymerization step performed at a pH between 4 and 6, and recovering and optionally purifying the monomers and/or oligomers, wherein said enzymatic depolymerization step is implemented in a reaction medium wherein the equivalent TA concentration in the liquid phase of said reaction medium is of at least 10 g/kg, preferably of at least 20 g/kg, more preferably of at least 30 g/kg based on the total weight of the liquid phase of the reaction medium and wherein at least 90%, preferably at least 95%, more preferably at least 96%, 97%, 98%, 99%, of the equivalent TA in the liquid phase of said reaction medium is in the form of salts.

All particular embodiments exposed above in connection with the process for degrading polyester containing material, such as plastic product, also apply to the methods of producing monomers and/or oligomers and to the methods of recycling.

### EXAMPLES

### Example 1—Process of Degrading a Plastic Product Comprising PET Comprising a Main Acidic Depolymerization Step and a Preliminary Enzymatic Depolymerization Step

Washed and colored flakes from bottle waste comprising 98% of PET with a mean value of crystallinity of 27% were foamed, by submitting the flakes (98.5% by weight based on the total weight of the mix introduced in the extruder) to an extrusion with 1% by weight of citric acid (Orgather exp 141/183 from Adeka) and 0.5% by weight of water, based on the total weight of the mix introduced in the extruder, in a twin-screw extruder Leistritz ZSE 18 MAXX at a temperature above 250° C. The resulting extrudate was granulated into 2-3 mm solid pellets with a crystallinity level of 7% (i.e., foamed PET).

The degrading process of the invention (including a preliminary depolymerization step and a main depolymerization step) was carried out in 500 mL reactors using a variant of LC-Cutinase (Sulaiman et al., Appl Environ Microbiol. 2012 March). Such variant (herein after “LCC-ICCIG”) corresponds to the enzyme of SEQ ID No 1 with the following mutations F208I+D203C+S248C+V170I+Y92G as compared to SEQ ID NO:1, and was expressed as recombinant protein by Trichoderma reesei.

At the beginning of the process, foamed PET was added in the reactor at a concentration of 200 g/kg based on the total weight of the initial reaction medium and LCC-ICCIG was added at 4 mg/g PET in 100 mM phosphate buffer pH 8 (except for Ref-2 wherein PET and enzyme are added in water). During the preliminary depolymerization step, the temperature was regulated at 60° C. and the pH of the reaction medium was regulated at pH 8±0.05 by addition of NaOH solution at 25% (Ref-1, Ref-4, Ref-5) or 5% (Ref-2, Ref-3).

The pH regulation and conditions during the preliminary depolymerization step were maintained until the equivalent TA concentration in the liquid phase of the reaction medium reached specific values between 33 and 90 g/kg as referenced in the Table 1 below (“Switch equivalent TA concentration” linked with a “Switch depolymerization rate”). Then the addition of base was stopped (“Switch NaOH addition quantity” also referenced in Table 1) and the temperature was decreased to 56° C. Accordingly, the pH of the reaction medium decreased until it reached the target pH for the main depolymerization step, as also referenced in Table 1 below.

During the preliminary depolymerization step, the equivalent TA concentration in the liquid phase was measured via regular sampling. Samples from the reaction medium were analyzed by Ultra High Performance Liquid Chromatography (UHPLC) for measuring the amount of equivalent terephthalic acid produced.

The samples were diluted in 100 mM potassium phosphate buffer, pH 8. One mL of samples or diluted samples were mixed with 1 mL of methanol and 100 μL of 6 N HCl. After homogenization and filtration through a 0.45 μm syringe filter, 20 μL of sample were injected into the UHPLC, Ultimate 3000 UHPLC system (Thermo Fisher Scientific, Waltham, MA) including a pump module, a sampler automatic, a column thermostated at 25° C. and a UV detector at 240 nm. The molecule of terephthalic acid and oligomers (MHET and BHET) were separated using a gradient of methanol (30% to 90%) in 1 mM H₂SO₄ at 1 m/min through a HPLC Discovery HS C18 column (150 mm×4.6 mm, 5 μm) equipped with a precolumn (Supelco, Bellefonte, PA). TA alone, MHET and BHET were measured according to standard curves prepared from commercially available TA and BHET and internally synthesized MHET (by partial base-catalyzed hydrolysis of BHET). The TA equivalent is the sum of the measured TA, MHET and BHET.

During the main depolymerization step, the PET depolymerization rate was determined by the measure of the total equivalent TA production (both soluble and precipitated TA). Said production was determined by the quantification of TA in the total slurry fraction (including the liquid phase and further containing precipitated TA in suspension in this liquid phase) using the method described for the preliminary depolymerization step, said method enabling the dissolution of precipitated TA.

The depolymerization rate after 140 h of reaction and the pH of the reaction medium during the main depolymerization step are disclosed in Table 1 below. Table 1 also references the equivalent TA concentration at which the pH regulation in the preliminary depolymerization step was stopped, as well as the equivalent TA concentration in the reaction medium (and the base concentration added in the reaction medium) before the main depolymerization step.

Two controls were also performed:
“Control 1” corresponding to a process performed at 56° C. in 100 mM phosphate buffer wherein the pH was regulated at 8 by the addition of NaOH solution at 25%.
·“Control 2” corresponding to a process performed at 56° C. in 100 mM phosphate buffer wherein the pH was regulated at 5.2 by the addition of NaOH solution at 5%.

After 140 h of reaction, the theoretical base consumption (Y base) was determined and corresponds to the base quantity added in the final reaction medium in order to solubilize the precipitated TA (or to the base quantity that should have been introduced if the whole process would have been implemented at pH 8 with the same enzyme). The base consumption saving (in %) during said process was then determined by the following formula:

$$base consumption saving = 100 - \frac{total base consumption \times 100}{Y base}$$

TABLE 1
Parameters and results of processes of the invention

| | | Switch | | | | |
|---|---|---|---|---|---|---|
| | | NaOH | | | pH during | |
| | Switch equivalent | addition in | | Total | the main | |
| | TA concentration | the reaction | Switch | depolymerization | acidic | Base |
| | in the liquid | medium | depolymerization | rate at | depolymerization | consumption |
| | phase (g/kg) | (g/kg) | rate (%) | T = 140 h (%) | step | saving (%) |
| Ref-1 | 38 | 16 | 21% | 39% | 5.26 | 47% |
| Ref-2 | 33 | 12 | 21% | 35% | 5.27 | 44% |
| Ref-3 | 36 | 13 | 21% | 35% | 5.35 | 43% |
| Ref-4 | 58 | 24 | 33% | 62% | 5.42 | 47% |
| Ref-5 | 90 | 38 | 56% | 92% | 5.42 | 39% |
| Control 1 | — | — | — | 95% | 8 | 0 |
| (regulation | | | | | | |
| pH 8) | | | | | | |
| Control 2 | — | — | — | 38% | 5.2 | 25% |
| (regulation | | | | | | |
| pH 5.2) | | | | | | |

The results show that the process of the invention allows base savings between 39% and 47% as compared to a regulated process at pH 8 (Control 1, no base consumption saving), or to a regulated process at pH 5.2 (Control 2, 25% base saving).

### Example 2—Degradation Process of a PET Plastic Product Comprising a Preliminary Enzymatic Depolymerization Step Followed by a Fed Batch Acidic Depolymerization Step

The process of the invention was implemented using the conditions used for Ref-5 of Example 1.

When 90% of the PET previously introduced was hydrolyzed (=136 h), additional PET and enzymes were added in order to reach a total added PET amount of 400 g/kg (based on the total weight of the initial reaction medium), and to maintain an enzyme concentration of 4 mg per g of PET as described in the following Table 2. The total equivalent concentration of polyester is given in relation to the total weight of the initial reaction medium.

TABLE 2
Parameters of the process of the invention

| | | |
|---|---|---|
| Time (hours) | 0 | 136 |
| Total concentration of polyester in the reaction medium | 200 | 400 |
| (g of polyester introduced during the process/kg of the | | |
| initial reaction medium) | | |
| Enzyme quantity in the initial reaction medium in mg per | 4 | 4 |
| g of polyester | | |

The depolymerization rate after 350 h and the base consumption saving were 70% and 60%, respectively.

### Example 3—Process of Degrading a PET Plastic Product Comprising a Preliminary Enzymatic Depolymerization Step and a Main Depolymerization Step with a PETase and a MHETase

The beginning of the process of the invention was implemented as described in Example 1 (i.e flakes used, enzyme and quantity thereof).

During the preliminary depolymerization step, the temperature was regulated at 60° C. and the pH of the reaction medium was regulated at pH 8±0.05 by addition of NaOH solution at 25%.

The pH regulation and conditions during the preliminary depolymerization step were maintained until the equivalent TA concentration in the liquid phase of the reaction medium reached 49.3 g/kg (i.e after 9.1 h of reaction). Then, the addition of base was stopped, and the temperature was decreased to 56° C.

After 23.4 h of reaction, 9.5 mg of purified MHETase of Ideonella sakaiensis of SEQ ID No 2, expressed by E. coli, were added to the reaction medium.

The equivalent TA concentration at which the pH regulation in the preliminary depolymerization step was stopped, the equivalent TA concentration in the reaction medium (and the base concentration added in the reaction medium) before the main depolymerization step as well as the depolymerization rate after 70 h of reaction and the pH of the reaction medium during the main depolymerization step are disclosed in Table 3 below.

Ref-4 of Example 1 can be considered as a control wherein no MHETase has been added (control-3).

TABLE 3
Parameters and results of processes of the invention

| | | Switch | | | | |
|---|---|---|---|---|---|---|
| | | NaOH | | | | |
| | Switch equivalent | addition in | | Total | pH during | |
| | TA concentration | the reaction | Switch | depolymerization | the main | Base |
| | in the liquid | medium | depolymerization | rate at | depolymerization | consumption |
| | phase (g/kg) | (g/kg) | rate (%) | T = 70 h (%) | step | saving (%) |
| Ref-6 | 49.3 | 20 | 30% | 69% | 5.4 | 59% |
| Ref-4 | 58 | 24 | 33% | 58% | 5.42 | 43% |
| (=control 3) | | | | | | |

After 70 h, the depolymerization rate and the base consumption saving of Ref-6, as compared to a regulated process at pH 8, were 69% and 59%, respectively.

These results further show that MHETase addition improves the depolymerization rate as well as the base consumption saving as compared to the process of the invention without MHETase.

### Example 4—Process of Degrading a PET Plastic Product Comprising a Preliminary Enzymatic Depolymerization Step and a Main Depolymerization Step with a PETase

The degrading process of the invention (including a preliminary depolymerization step and a main depolymerization step) was carried out in a 500 mL reactor using a purified variant of the enzyme of SEQ ID No 3 containing the following mutations L210T+V172I+N213M and expressed as a recombinant protein by E. coli.

The flakes introduced and the conditions of the preliminary depolymerization step (i.e pH regulation, temperature, enzyme quantity) were the same as described in Example 3.

The pH regulation and conditions during the preliminary depolymerization step were maintained until the equivalent TA concentration in the liquid phase of the reaction medium reached 42 g/kg. Then, the addition of base was stopped, and the temperature was decreased to 56° C.

The equivalent TA concentration at which the pH regulation in the preliminary depolymerization step was stopped, the equivalent TA concentration in the reaction medium (and the base concentration added in the reaction medium) before the main depolymerization step as well as the depolymerization rate after 30 h of reaction and the pH of the reaction medium during the main depolymerization step are disclosed in Table 4 below.

TABLE 4
Parameters and results of process of the invention

| | | Switch | | | | |
|---|---|---|---|---|---|---|
| | | NaOH | | | | |
| | Switch | addition in | | | | |
| | equivalent TA | the | | | | |
| | concentration in | reaction | Switch | Total | pH during the main | Base |
| | the liquid phase | medium | depolymerization | depolymerization | depolymerization | consumption |
| | (g/kg) | (g/kg) | rate (%) | rate at T = 30 h (%) | step | saving (%) |
| Ref-7 | 42 | 21 | 27% | 35% | 5.1 | 23% |

The depolymerization rate after 30 h and the base consumption saving compared to a regulated process at pH 8 were 35% and 23%, respectively.

1-25. (canceled)

26. A process for degrading a plastic product comprising at least one polyester comprising at least a terephthalic acid monomer (TA) wherein the process comprises a main step of enzymatic depolymerization of said at least one polyester performed at a pH between 4 and 6, and wherein said enzymatic depolymerization step is implemented in a reaction medium wherein the equivalent TA concentration in the liquid phase of said reaction medium is of at least 10 g/kg based on the total weight of the liquid phase of the reaction medium with at least 90% of said equivalent TA in the form of salts.

27. The process according to claim 26, wherein the main step of enzymatic depolymerization is implemented in a reaction medium wherein the equivalent TA concentration in the liquid phase of the reaction medium is comprised between 20 g/kg and 80 g/kg and wherein at least 95% of the equivalent TA in the liquid phase of said reaction medium is in the form of salts.

28. The process according to claim 26, wherein the pH of the main enzymatic depolymerization step is not regulated.

29. The process according to claim 26, wherein the main enzymatic depolymerization step is implemented at a pH between 4 and 5.5 and/or wherein the main enzymatic depolymerization step is implemented at a temperature between 40° C. and 80° C.

30. The process according to claim 26, wherein the main enzymatic depolymerization step is implemented at a pH between 5.0 and 5.5, and wherein the equivalent TA concentration in the liquid phase of the reaction medium is between 5 g/kg and 110 g/kg.

31. The process according to claim 26, wherein the main enzymatic depolymerization step is performed by contacting the plastic product in the reaction medium with an enzyme able to degrade said at least one polyester.

32. The process according to claim 26, wherein the reaction medium is obtained by implementing a preliminary depolymerization step, prior to the main enzymatic depolymerization step, said preliminary depolymerization step being implemented by contacting the plastic product in an initial reaction medium with a depolymerizing agent selected from chemical and/or biological depolymerizing agents, optionally with at least an enzyme able to degrade said polyester, said preliminary depolymerization step being implemented at a pH between 6.5 and 10.

33. The process according to claim 32, wherein the preliminary depolymerization step is implemented by contacting the plastic product in an initial reaction medium with at least an enzyme able to degrade said polyester of the plastic product and wherein the pH is regulated at a pH between 7.00 and 9.50 by addition of a base in the reaction medium, and/or wherein the temperature is between 50° C. and 80° C.

34. The process according to claim 33, wherein the pH of the preliminary depolymerization step is regulated at the given pH until the equivalent TA concentration in the liquid phase of the reaction medium is between 5 g/kg and 110 g/kg and wherein the main depolymerization step is implemented at a pH between 5 and 5.5.

35. The process according to claim 32, wherein both depolymerization steps are implemented by contacting the plastic product with at least one enzyme exhibiting a polyester-degrading activity at pH between 4 and 9 and/or with at least two enzymes.

36. The process according to claim 35, wherein the plastic product is contacted simultaneously with one or more PETase and one or more MHETase.

37. The process according to claim 36, wherein the PETase and the MHETase are included in a multienzyme system.

38. The process according to claim 35, wherein the plastic product is contacted first with the PETase, and the MHETase is introduced in the reaction medium after the PETase.

39. The process for degrading a plastic product comprising at least one polyester comprising at least a terephthalic acid monomer (TA) according to claim 32, comprising:
(a) a preliminary enzymatic depolymerization step implemented at a given pH regulated between 7.5 and 8.5 and at a temperature between 60° C. and 72° C.; and
(b) a main depolymerization step implemented at a pH between 5.0 and 5.5 wherein the pH is not regulated and at a temperature between 50° C. and 65° C.,
wherein each depolymerization step comprises contacting the plastic product with at least one enzyme able to degrade the at least one polyester, and wherein the pH regulation of step (a) is stopped as the equivalent TA concentration in the liquid phase of the reaction medium is between 5 g/kg and 110 g/kg based on the total weight of the liquid phase of the reaction medium.

40. The process for degrading a plastic product comprising at least one polyester comprising at least a terephthalic acid monomer (TA) according to claim 39, comprising:
(a) a preliminary depolymerization step implemented at a given pH regulated between 7.5 and 8.5 and at a temperature between 60° C. and 72° C., by contacting the plastic product with at least one PETase; and
(b) a main depolymerization step implemented at a pH between 5.0 and 5.5, wherein the pH is not regulated and at a temperature between 50° C. and 65° C., by contacting the plastic product with at least one PETase and optionally at least one MHETase,
wherein the pH regulation of step (a) is stopped as the equivalent TA concentration in the liquid phase of the reaction medium is comprised between 30 g/kg and 95 g/kg.

41. The process for degrading a plastic product comprising at least one polyester comprising at least a terephthalic acid monomer (TA) according to claim 39, comprising:
(a) a preliminary depolymerization step implemented at a given pH regulated between 7.5 and 8.5 and at a temperature between 60° C. and 72° C. by contacting the plastic product simultaneously with at least one PETase and at least one MHETase; and
(b) a main depolymerization step implemented at a pH between 5.0 and 5.5, wherein the pH is not regulated and at a temperature between 50° C. and 65° C.,
wherein the pH regulation of step (a) is stopped as the equivalent TA concentration in the liquid phase of the reaction medium is comprised between 30 g/kg and 95 g/kg.

42. The process for degrading a plastic product comprising at least one polyester comprising at least a terephthalic acid monomer (TA) according to claim 39, wherein at least one additional amount of PETase and/or MHETase is added once or several times to the reaction medium during the main depolymerization step (b).

43. The process according to claim 26, wherein the equivalent TA concentration in the reaction medium is obtained by addition of TA salts and/or oligomer salts and/or by addition of TA in its acid form and base, in a reaction medium prior to the main depolymerization step until reaching an equivalent TA concentration in the liquid phase of the reaction medium between 10 g/kg and 80 g/kg based on the total weight of the liquid phase of the reaction medium, with at least 90% of the equivalent TA in the liquid phase in the form of salts, and wherein the main depolymerization step is implemented at a pH between 5 and 5.5.

44. The process according to claim 26, wherein the concentration of polyester introduced in the reaction medium before the main depolymerization step or before to the preliminary depolymerization step is above 150 g/kg based on the total weight of the reaction medium.

45. The process according to claim 26, wherein the polyester is selected from PET, PTT, PBT, PEIT, PBAT, PCT, PETG, PBST and PBSTIL.

46. The process according to claim 26, wherein said polyester is submitted to an amorphization and/or a foaming step prior to the main depolymerization step or prior to the preliminary depolymerization step and/or wherein the process further comprises a step of recovering and optionally purifying the oligomers and/or monomers resulting from the depolymerization of said polyester.

47. The process according to claim 26, wherein a PETase is used in the preliminary depolymerization step and/or in the main depolymerization step and is selected from an enzyme having at least 75% identity to the full length amino acid sequence set forth in SEQ ID NO: 1 and/or to the full length amino acid sequence set forth in SEQ ID NO: 3.

48. The process according to claim 26, wherein a MHETase is used in the preliminary depolymerization step and/or in the main depolymerization step and is selected from an enzyme having at least 75% identity to the full length amino acid sequence set forth in SEQ ID NO: 2.

49. A method for producing TA from a plastic article containing at least one polyester having at least one TA monomer comprising submitting the plastic article to a main enzymatic depolymerization step performed at a pH between 4 and 6, and recovering and optionally purifying the monomers and/or oligomers, wherein said enzymatic depolymerization step is implemented in a reaction medium wherein the equivalent TA concentration in the liquid phase of said reaction medium is of at least 10 g/kg based on the total weight of the liquid phase of the reaction medium and wherein at least 90% of the equivalent TA in the liquid phase of said reaction medium is in the form of salts.

50. A reaction medium suitable to be used in a degradation process of a plastic product comprising at least one polyester comprising at least one monomer of terephthalic acid (TA), comprising at least 10 g/kg of equivalent TA in the liquid phase of the reaction medium based on the total weight of the liquid phase of the reaction medium with at least 90% of said equivalent TA in the form of salts, and optionally at least one enzyme able to degrade a polyester.


SEQUENCE LISTING (3 sequences)

SEQ ID NO: 1 - protein, 258 aa, artificial sequence
  cutinase
SNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELDNASHFAPNSNNAAISVYTISWMKLWVDNDTRYRQFLCNVNDPALSDFRTNNRHCQ

SEQ ID NO: 2 - protein, 594 aa, ARTIFICIAL SEQUENCE
  MHETase Ideonella
CAGGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFATIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPPLEHHHHHH

SEQ ID NO: 3 - protein, 267 aa, ARTIFICIAL SEQUENCE
  polyester degrading enzyme
MANPYERGPDPTESSIEAVRGPFAVAQTTVSRLQADGFGGGTIYYPTDTSQGTFGAVAISPGFTAGQESIAWLGPRIASQGFVVITIDTITRLDQPDSRGRQLQAALDHLRTNSVVRNRIDPNRMAVMGHSMGGGGALSAAANNTSLEAAIPLQGWHTRKNWSSVRTPTLVVGAQLDTIAPVSSHSEAFYNSLPSDLDKAYMELRGASHLVSNTPDTTTAKYSIAWLKRFVDDDLRYEQFLCPAPDDFAISEYRSTCPFLEHHHHHH